(19) United States
(12) Patent Application Publication
(10) Pub. No.: US 2009/0053282 A1
(43) Pub. Date: Feb. 26, 2009

(54) Bone Grafting Method and Kit

(76) Inventors: Dennis G. SMILER, Encino, CA (US); Muna SOLTAN, Riverside, CA (US)

Correspondence Address: CROWELL & MORING LLP;INTELLECTUAL PROPERTY GROUP, P.O. BOX 14300, WASHINGTON, DC, 20044-4300, US

(21) Appl. No.: 12/196,709

(22) Filed: Aug. 22, 2008

Related U.S. Application Data
(60) Provisional application No. 60/957,659, filed on Aug. 23, 2007.

(51) Int. Cl. A61B 19/02; C12N 5/02; A61F 2/28

(52) U.S. Cl. 424/423; 435/378; 206/572

(57) ABSTRACT

Bone grafting methods involving harvesting at least of the following types of cells: osteoblasts, adult stem cells and primitive mesenchymal cells and applying these cells to a site intended for a bone graft in combination with the application of a suitable matrix material, where the matrix material is an allograft, xenograft or alloplast matrix material or a combination thereof.

The present application claims priority under 35 U.S.C. § 119 to U.S. Provisional Patent Application No. 60/957,659, filed Aug. 23, 2007 the entire contents of which is herein expressly incorporated by reference.

### FIELD OF THE INVENTION

The present invention, in certain embodiments, relates to bone grafting methods. More particularly, the invention of grafting a combination of bone marrow aspirated stem cells mixed with either an allograft (demineralized bone, undemineralized bone), xenograft (bovine resorbable hydroxyapatie) or alloplast (β-tricalcium phosphate, calcium sulfate and β-tricalcium sulfate) as well as mixtures of the foregoing enables repair and reconstruction of osseous defects throughout the body. This procedure has efficacy in orthopedic surgery, spine surgery, and oral and maxillofacial defects of maxilla and/or mandible. In other embodiments, a kit for performing the disclosed methods is provided.

### SUMMARY OF THE INVENTION

For simplicity and illustrative purposes, the principles of the present invention are described by referring to various examples. One of ordinary skill in the art will readily recognize that the same principles are equally applicable to, and can be implemented in other forms, and that any such variation would be within those modifications that do not part from the true spirit and scope of the present invention. The invention is not limited in its application to the details of any particular formulation shown, since the invention is capable of other embodiments. The following examples are provided for illustrative purposes and do not and should not be understood to limit the claims appended hereto. The terminology used herein is for the purpose of description and not of limitation.

With appropriate selection and application of graft materials for a given defect site, the original morphology of the site can be regained or even surpassed. Selection of an inappropriate graft material may lead to resorption of the graft material or failure of the graft to integrate with the surrounding tissue. Alternatively, disrupted or lost tissue may be replaced with fibrous tissue rather than functioning bone.

Bone regeneration by using cell-based strategies is related to the potentials of bone marrow adult stem cells as a regenerative source for bone and other tissues. This new standard for bone grafting is emerging as an alternative to autogenous bone grafts. Bioengineered graft materials that provide a scaffold to support the proliferation, differentiation, and maturation of the stem cells as well as facilitating angiogenesis, go hand in hand for a consistent and predictable result.

The combination of bone marrow aspirate graft techniques and an appropriate graft scaffold matrix of either an allograft, xenograft, or alloplast, or a combination thereof presents a methodology yielding a bone-graft material that is most likely to be transformed into viable new bone, and ensures that bone regeneration results, as opposed to fibrous repair.

For successful bone grafts, four main elements should be considered: the blood supply to the site must be sufficient to nourish the graft; a resorbable matrix should be provided, the graft should be stabilized during healing, and bone-forming cells (osteoblasts) should be present at the site. These elements are of particular relevance to bone grafts of the maxilla and mandible. Soluble regulators such as cytokines are acquired from the blood. These materials may supplemented to promote and facilitate the desired osteogenesis, including osteoinduction, osteoconduction and any desired osteointegration. Resorbable matrices are commercially available for surgical applications. Suitable stabilization of the matrix can be achieved with guided resorbable membranes, titanium mesh, bone tacks, screws and other suitable fastening or holding devices and methods. Another important component for a successful bone graft are the cells. In particular, it is desirable that osteoblasts populate the matrix in sufficient quantity to form bone.

### Cells

Without osteoblasts or precursor cells, bone generally will not form. In one aspect, the invention relates to a process by which to obtain adult stem cells from bone marrow aspirate. These stem cells differentiate into osteoblasts. Primitive mesenchymal cells may be brought to the recipient site from harvested bone marrow aspirate from, for instance, the iliac crest. These cells populate the extracellular matrix and transform into osteoblasts.

Bone tissue is composed of cells, insoluble extracellular matrix, and soluble molecules that serve as regulators for cell function. The bone cells may be one of three types: osteocytes, osteoblasts, or osteoclasts. Osteocytes are the mature bone cells found within lacunae of the dense cortical bone. A solid mass containing only microscopic channels, cortical bone contains a significant number of either osteoblasts, which are responsible for bone apposition, or osteoclasts, involved with remodeling and bone resorption. The periosteum that is tightly attached to the outer surface of cortical bone does contain the potential for both osteoblastic and osteoclastic activity, along with collagen fibers. An even larger supply of osteoblasts and osteoclasts is found within the spongy cancellous bone that lies beneath the cortical bone. Cancellous bone is composed of a lattice of large hydroxyapatite plates and rods, known as the trabeculae. Because cancellous bone has an eight times higher surface to bone ratio than cortical bone, this provides higher access to bone-forming cells.

Osteoblasts create new bone. For a successful graft, the graft matrix must be populated by osteoblasts or primitive mesenchymal cells that can be transformed into osteoblasts. If osteoblasts are not present in the recipient bed, they must be harvested and brought to the site. Alternatively, primitive mesenchymal cells may be brought in via the blood supply from the adjacent bone or periosteum to populate the extracellular matrix and be transformed into osteoblasts. The absence of a sufficient population of osteoblasts will likely cause the graft to fail.

Bone is encircled by periosteum of dense connective tissue that contributes to the generation of osteoblasts. It has two layers: an outer fibrous layer with typical fibroblasts; and an inner cellular layer, which contains osteoprogenitor cells that are capable of contributing to osteoblasts. In addition, a layer of cells called the endosteum (endosteal cells) lines the marrow surface of compact bone. Like the periosteal cells, these endosteal cells are also osteoprogenitor cells capable of becoming osteoblasts.

Osteoblasts and/or adult stem cells or primitive mesenchymal cells may also be present in the cancellous compartment of a recipient site. Osteoblasts may also be found in adjacent decorticated bone, harvested autogenous bone, circulating blood, or bone marrow aspirate. If osteoblasts are not present at the recipient site, they are preferably harvested as a graft material. Without osteoblasts, the likelihood of graft failure is significantly increased.

Hematopoietic stem cells augment the limited number of available stem cells to support angiogenesis and vasculogenesis. Osteoblastic precursors can differentiate into the mature osteoblasts that are needed to promote osteogenesis. Marrow cells promote osteogenesis.

By definition, stem cells are capable of both self-renewal and differentiation into a mature cell type. Stem cells divide to form one daughter cell that goes on to differentiate and one daughter cell that retains its stem cell properties. The classification of cells as stem cells is based on their species of origin, tissue of origin, or differentiation capability of greater than one specific type of the mature cells. Some stem cells are more pluripotent than others. For example, all cells within the early embryo are totipotent up until the 16-cell stage (or thereabout) and are thought to be the only single cells capable of differentiating into any cell type. Adult stem cells are pluripotent but have more limited differentiation ability and, thus, are considered multipotent. Multipotent stem cells are only committed to differentiate to a limited number of types of cells that have a specific function (e.g., cells that contribute to all the cells of the blood [hematopoietic stem cells] and other committed stem cells, such as mesenchymal stem cells). Mesenchymal stem cells are multipotent, reside in the bone marrow of adult human beings, and have differentiated into bone, fat, muscle, cartilage, and neurons.

Bone marrow is found in the center of large flat bones and can be transplanted. Bone marrow contains abundant adult stem cells. Recent studies have shown that adult stem cells are more plastic than previously thought. The term plasticity refers to the ability of adult stem cells to cross lineage barriers and adopt the expression and function of other cell types. Should adult stem cells hold the same clinical potential of embryonic stem cells, it would allow researchers to bypass the ethical and practical issues related to the preparal use of embryonic stem cells.

Bone marrow-derived stem cells include hematopoietic stem cells, marrow stromal cells (mesenchymal stem cells), and multipotent adult progenitor cells. Bone marrow represents the main source of mesenchymal stem cells. The hematopoietic cells are irreversibly committed toward a blood lineage, but other stromal cells can differentiate to form adipocytes, chondrocytes, osteoblasts, and other connective tissue cells. Therefore, transplantation of marrow cells contributes to hematopoietic and osteogenic cells. A central issue concerning bone formation relates to the developmental lineages of osteoblasts and osteoclasts. Osteoblasts derived from mesenchymal cells present bone in the skeletal environment. Osteoclasts are derived from blood-borne monocyte/macrophage cells.

Living cells, particularly bone marrow cells, make cellular contributions to bone formation. Marrow cells promote osteogenesis. Bone marrow contains osteoblast precursors that can differentiate into the mature osteoblasts that are needed to promote osteogenesis. Developing a method and technique to harvest bone marrow and its osteoblastic precursor cells, and, subsequently, implant them into sites of impaired bone healing or bone-graft matrix affords a new approach for bone regeneration.

The delivery of pluripotent mesenchymal stem cells within a resorbable matrix to induce osteogenesis has been successful. The evidence that bone marrow fosters successful grafts is compelling and indicates that significant bone formation occurs when marrow is implanted in osseous defects.

Suitable stem cells, for instance, mesenchymal stem cells may be harvested from bone marrow, or, alternatively, from peripheral blood.

### Blood Supply

A sufficient blood supply to nourish the graft must be present. The optimal result of bone grafting is regeneration, as opposed to repair. Repair is the process in which the continuity of lost tissue is regained by new tissue that does not restore the structure and function of the original tissue. On the other hand, regeneration is the biologic process by which the structure and function of the original tissues are regained. One important factor that influences the course of regeneration versus repair is the blood supply available to the graft and surrounding tissues.

In tissues that lack an adequate blood supply, cells will not remain viable, and regenerative healing will not occur. Furthermore, without an adequate vascular supply, a fibrin clot may not form. The fibrin clot serves as the initial matrix where the mesenchymal cells can migrate, divide, and develop into osteoblasts. It also serves as an anchorage for the osteoblasts that will continue the bone-forming process.

### Graft Stabilization

The graft environment also influences whether regeneration or repair will occur. Mechanical stresses on the healing graft may lead to excessive distortion and disruption of the initial fibrin clot. Regeneration then decreases, and a lower type of repair tissue, such as fibrous tissue, forms. For example, grafting an osseous defect with loss of buccal bone, if the graft is not stabilized to prevent movement, it is likely that fibrous tissue will fill the defect instead of bone. The surgeon can control this factor by ensuring the stability of the matrix during the healing process. Using fixation devices such as guided bone regeneration (GBR) membranes, titanium mesh, bone screws, or bone tacks, depending upon the surgery being per-formed, complete this process.

For Oral Applications, the Flap Must be Sutured without Tension on the Incision

Primary closure over the graft ensures tissue continuity over the graft, which in turn increases the blood sup-ply to the graft. It also protects the graft from external forces, as well as the salivary enzymes and other elements in the oral environment. To avoid the risk of the incision opening, tension must not be placed on the incision line when achieving primary closure.

The periosteum is not elastic and will not stretch over incision sites when grafts are added. One way to achieve primary closure without placing the incision under tension is by using a scalpel to cut and spread the periosteum only. Care must be taken to avoid any intrusion upon the under-lying connective tissue and blood vessels. Thus, the periosteum can be spread and extended. This technique has consequences that must be considered in any bone grafting procedure. For one thing, any incision into the periosteum reduces its effectiveness in contributing to bone healing and also allows for fibrovascular invasion into the graft. To prevent this result, the surgeon should use a GBR membrane between the graft and periosteum. However, the use of such membranes also reduces the effectiveness of the periosteum in bone healing.

Choosing the right membrane is critical to the outcome of regenerative therapy. Because bone is the slowest growing tissue, the GBR membrane must be cell occlusive, keeping faster growing tissues like epithelium, fibrous tissue, or gingival connective tissue out of the defect space and al-lowing osseous tissue to form. The decision to use a resorbable or nonresorbable membrane is based on the size and location of the defect, how long does the membrane need to form as a barrier, and how much bone re-generation is required. A rule of thumb is 1 mm of bone regeneration per month per barrier function time. For example, a 2-3-month barrier function time is required for small defects of 2-3 mm, and larger defects may require 6-13 months.

### Successful Bone Grafting

Successful bone grafting requires addressing a number of questions. If the questions are answered correctly, the graft will likely succeed. The key questions that must be addressed, include

What is the quality of the recipient bone at the graft site?

What type of material should be grafted?

How much recipient bone is present?

How should it be grafted?

### Bone Quality

One must evaluate what kind of bone is available at the site. Is it predominantly cortical, corticocancellous, or cancellous? It has been estimated that cells within the cancellous bone are responsible for at least 60% of a patient's bone healing capacity. The periosteum in a young, healthy patient may contribute an additional 30% of bone healing, with the osteocytes within the cortical compartment responsible for approximately 10% of bone healing. However, as the patient ages, the effectiveness of the periosteum to regenerate bone is decreased.

The function of alveolar bone is to stabilize teeth. When teeth are extracted or removed, the bone resorbs and, more-over, the proportion of cancellous bone shrinks, relative to the cortical bone at the site. As the cancellous compartment decreases, the reservoir for osteoblasts does likewise. Computerized tomography will reveal the ratio of cancellous bone-to-cortical bone at the recipient bed before surgery.

If the recipient bed is found to consist primarily of cortical bone, sufficient osteoblasts will not be present to ensure new bone formation. Bone that is rich in osteoblasts, either cancellous or corticocancellous autogenous bone, must then be harvested and used as a graft material. On the other hand, the presence of a large cancellous compartment at the graft site will allow the surgeon to use a scaffold matrix other than autogenous bone. The recipient site must be decorticated to allow the cancellous bone to provide the supply of osteoblasts. An exception is the sinus, where no decortication is performed, but the precursor cells for the oteoblasts are found in the circulating blood.

A common condition at graft sites is the presence of corticocancellous bone. In such cases, the surgeon must evaluate the ratio of cortical to cancellous bone present. If cortical bone is most predominant, autogenous bone will optimize bone graft success. If the bone is mostly cancellous, a combination of graft materials (e.g., xenograft, allograft, and alloplast) or autogenous bone can be effective. The preference of the authors is to use resorbable and natural materials.

Blood flow to the graft is crucial. This process is important even when performing a small graft, such as a socket-preservation graft after an ex-traction. Often when a tooth is extracted for periodontal disease, the tooth may be removed without provoking any bleeding. If a graft material is placed within the socket, the surgeon might discover after 3-5 months that bone has not regenerated. Such failures can often be explained by the surgeon's not considering that blood flow is necessary to nourish the graft and populate it with bone-forming cells. Even when performing a simple socket-preservation technique after tooth removal, the surgeon must remove the thin dental lamina lining the socket to tap into the cancellous bone. Likewise, the graft material must be packed loosely into the site to allow spacing between the particles for angiogenesis and revascularization within the graft.

### Bone Quantity

Once the best graft material option for a given site has been identified, the surgeon can move on to consider the best choice among several surgical techniques. The bone quantity at the recipient site determines which surgical technique can be considered. All surgical techniques for bone grafting fall into two main categories: onlay grafts and interpositional grafts.

Onlay grafts involve the placement of graft material on top of the cortical bone. The graft material constitutes the topmost layer at the site and is covered by the periosteum. Either block or particulate onlay grafts may be used. Block grafts may be composed of either cortical or corticocancellous autogenous bone, or of a compressed xenographic or allographic material. Particulate onlay grafts may consist of cancellous or corticocancellous autogenous bone, allographic, xenographic, or alloplastic materials, or some combination thereof.

Interpositional grafts involve the placement of graft material within a 3, 4, or 5-walled cancellous compartment. These grafts include such techniques as the sinus-lift graft, in which the graft is placed within a defect bordered by the medial wall of the sinus, the alveolus, and posterior and anterior region of the sinus. Thus, the recipient site contains and stabilizes the graft material, and circulating blood flow provides cells, soluble regulators, and nourishment. Another interpositional graft is the split-cortical graft to the maxilla, in which the cortices are split vertically, exposing the cancellous compartment. Bone graft material is then placed within the fissure. A similar technique is the sandwich graft, used in the mandible. In this approach, a section of the cortical bone is removed, graft material is placed within the cancellous compartment, and then the plug of cortical bone is repositioned on top of the graft material and stabilized with bone screws. Another example of an interpositional technique is the socket graft.

### Stabilizing the Graft

Once the surgical technique and graft material have been chosen, the surgeon must consider the question of how the graft will be stabilized. In a 5-wall defect, the osseous walls stabilize the graft. No matter which surgical technique and graft material are used, if the graft is not stabilized, a lower form of repair (rather than bone regeneration) is likely to occur. The preferred options for graft stabilization are: a GBR membrane, a titanium-reinforced GBR membrane, titanium mesh, bone screws, or bone tacks.

In smaller defects, a GBR membrane can be used to stabilize the graft material. As the site to be grafted in-creases in size, a GBR membrane may not be sufficient to protect the graft from micromovement that would de-stabilize the matrix and disturb the blood clot. In this situation, a firmer stabilization material will be required, such as a combination of titanium mesh and a GBR membrane. This is especially true when the graft material is of a particulate composition.

If the donor bone is of a cortical or corticocancellous type, bone screws typically are used to stabilize the bone before it is covered with the periosteum and the mucoperiosteum is sutured. Whether particulate or corticocancellous, if the graft is not stabilized, then a lower form of healing will occur, and the bone graft will not be successful. The following case descriptions illustrate the use of this bone-grafting decision hiearchy.

### Case A

Extraction of the maxillary dentition resulted in 4 and 5-walled defects, primarily of the cancellous compartment. A decision was made to graft the ridge with a mixture of xenograft (PepGen P-15™; Dentsply Friadent Ceramed, Lakewood, Colo.) and freeze-dried demineralized bone allograft. The sockets were debrided and irrigated, and a minor alveoloplasty prepared the edentulous ridge for the interpositional graft. A GBR membrane was placed over the extraction sites, and the mucoperiosteal flap was repositioned and sutured.

### Case B

The bone quality of this edentulous maxilla was composed mostly of cancellous bone bordered by cortical bone. The presence of the cancellous bone made it possible to use a combination of xenograft material (PepGen P-15 228) and freeze-dried demineralized bone as graft materials. Because the ridge was severely resorbed, a decision was made to per-form a split-cortical interpositional graft. A surgical sagittal saw bisected the cancellous compartment and extended all the way to the sinus floor. Chisels spread the labial-buccal bone from the stable palatal bone. After placement of the graft material, a GBR membrane was placed. A split-thickness dissection of the labial mucosa left the periosteum on bone and permitted a nontension closure of the incision.

### Case C

The mandibular edentulous area had a small but adequate cancellous compartment bordered by dense cortical bone. A decision was made to augment the site using a combination of xenograft (PepGen P-15 228) and freeze-dried demineralized bone allograft. Trauma had reduced the site to a single wall of bone, and a decision was made to perform an interpositional (sandwich) graft. A wide-based flap exposed the site for preparation of vertical, inferior horizontal, and crestal sagittal bone cuts into the cancellous bone. The cortical shelf was removed, and the graft material was placed within the cancellous compartment. The cortical shelf was then replaced, and secured with a bone screw and GBR membrane.

### Case D

A quadrilateral osteotomy prepared the buccal wall for inward reflection. Careful elevation of the antral membrane exposed the superior aspect of the reflected buccal wall, medial wall of the sinus, alveolar bone, and posterior and anterior components of the sinus cavity. Thin cortical bone and a narrow cancellous compartment bordered the osseous defect. An interpositional graft composed of PepGen P-15 228 mixed with platelet-rich plasma reconstituted the buccal wall. For this interpositional graft, circulating primitive mesenchymal cells and preosteoblasts populated the PepGen P-15 228 matrix to form bone sufficient for the placement of implants.

### SUMMARY

The foregoing methodology provides a systematic approach to the series of decisions that must be made by implant practitioners before bone-augmentation graft surgery. The quality and quantity of recipient bone are first evaluated, and then the questions of what type of grafting material should be used and how it should be applied and stabilized are addressed. The presence of an adequate supply of osteoblasts and sustained vascularity is crucial to ensuring the success of the graft, as is stability of the graft during healing and tension-free primary closure of the incision.

Within this broad decision-making framework, more detailed decisions must be made. When nonautogenous materials are being used, a choice must be made as to which material will maximize the chances for graft success.

### Harvest Techniques

Unlike procedures for harvesting autogenous bone, the aspiration of bone marrow does not require an open surgical site. A relatively simple procedure with minimal morbidity, bone marrow aspirations can be performed in an outpatient setting, as is routinely performed by hematologists and oncologists. The bone marrow aspirant is combined with a matrix which is preferably nonautogenous and/or osteoconductive. This procedure offers a promising alternative to autogenous bone grafts. The large flat bones of the body are rich in red active marrow and are an excellent source of osteoprogenitor cells. Three locations are recommended for harvesting bone marrow from an adult patient: the anterior iliac crest, posterior ilium, and sternum.

Common problems associated with harvesting autogenous bone such as: arterial injury; ureteral injury; herniation; chronic pain; nerve injury; infection fracture; pelvic instability; cosmetic defects; hematoma; ileus and gait abnormality may be avoided using the present bone marrow aspiration process.

Generally, the process is performed as follows. Under sterile conditions, the site is identified and marked. The skin is then cleaned with iodine or chlorhexidine gluconate for patients who have iodine allergies. Local anesthetics are used in the conscious patient to infiltrate the epidermis, subcutaneous tissues, and periosteum. The same needle may be used to probe and confirm the precise location of the bone to be aspirated. Optionally, an anticoagulant such as heparin or a thrombin inhibitor maybe added to the syringe prior to aspiration.

A bone marrow aspiration/biopsy needle is then inserted up to the point of contact with the bone. Using steady pressure, and rotating the needle back and forth achieves penetration through the cortical bone. Decreased resistance indicates that entry into the marrow cavity has been achieved.

The first 2-3 ml of marrow aspirate contains the highest concentration of osteoprogenitor cells. With higher aspiration volumes, dilution of the marrow-derived cells by peripheral (venous) blood will occur. Therefore, the needle should be redirected or a new aspiration site selected if >2 ml of marrow aspirate is required. For repair of defects in the maxilla and mandible, >2 ml of bone marrow aspirate is not typically required.

In a healthy adult human being, aspiration of 2 ml of marrow can provide an average of 36 million (3.6×10⁷) marrow cells, of which 360 stem cells would be available for differentiation. An average ratio of 1 stem cell per 100,000 marrow cells is found in healthy individuals, with the highest majority of the marrow cells belonging to the various hematopoietic lineages. In vitro studies have shown that a mean of 2400 osteoblastic bone-forming cells producing alkaline phosphate positive colonies could be calculated. This number diminishes with age and in the presence of systemic disease. After the desired volume of marrow has been harvested, anticoagulation and filtration techniques may be implemented to concentrate the progenitor cells from the rest of the peripheral red blood cells as well as to collect the plasma, which includes cytokines and growth factors, as desired. Alternatively, or additionally, the bone marrow aspirate may be concentrated or separated by centrifuging, for instance for ten minutes. The desired fractions of the aspirate, for instance, the plasma with its cytokines and growth factors, as well as the progenitor cells, may then be used in the graft.

If centrifuging is performed, several distinct layers will become visible. At the bottom, the hematocrit or red blood cells, above that the myeloid erythroid layer appears as a buffy coat layer containg nucleated cells. Then the plasma layer and finally a fatty layer which is commonly yellow or milky in color. The plasma layer and the erythroid layer commonly vary in amounts, reflecting the quantity of blood aspirated with the marrow. Often the erythroid layer and the fatty layer are each about 1% of the total volume. The plasma and fatty layers can be removed using appropriate techniques, for instance suction with a pipette. The plasma layer includes cytokines and growth factors. The myeloid erythroid layer may be removed and the red blood cells discarded.

### Anterior Iliac Crest Bone Marrow Aspiration

The patient is lying in a prone position, and garments are removed to expose the anterior wing of the ilium. The border of the anterior wing is palpated. Palpation of the medial and lateral wall of the anterior ilium orients the site of needle puncture. The skin is stretched between 2 fingers over the bone crest identifying the thickness of the bone crest. The anterior position of the iliac crest and site of needle puncture can be outlined. The site is prepared with antiseptic, for instance, Betadine (Purdue Pharma L.P., Stamford, Conn.) solution, and an adhesive drape is placed over the aspiration site. Xylocalne (AstraZeneca Pharmaceuticals LP, Wilmington, Del.) local anesthesia is placed under the skin. Alternatively, another suitable local anesthetic may be provided. A longer needle is used to identify the midpoint of the iliac crest and deposit 3-4 mL 2% Xylocalne or another suitable anesthetic under the periosteum. The anesthetic may be buffered with a suitable solution such as sodium bicarbonate, if desired.

A “J” needle (Jamshidi Bone Mar-row Biopsy and Aspiration Tray; Cardinal Health, McCaw Park, Ill.) or other suitable needle is inserted, preferably by hand, through the skin into the anterior/posterior iliac wing (FIG. 3). The needle is rotated gently into approximately 1 cm of the marrow cavity. The stylet is removed from the needle and a 5-cc (or other appropriately sized) syringe attached. Bone marrow is aspirated by retraction of the plunger of the syringe. After 2-3 mL of marrow is collected, the needle may be repositioned if more marrow can be obtained, if necessary. The marrow is aspirated with a glass syringe in 3-5-cc aliquots with repositioning of the needle after each aspiration. This procedure is performed to ensure that marrow is aspirated rather than venous blood. The syringe is re-moved from the needle, and the needle is removed from the marrow space with an upward twisting motion. Pressure is placed over the aspiration site for 5 minutes, and a bandage is placed.

Case No. 1. The bone marrow aspirate was mixed with resorbable matrix in a glass syringe. After sinus-lift surgery to create the graft recipient site, the graft is deposited with loose compaction to reconstitute the buccal wall of the maxilla.

### Posterior Iliac Crest Bone Marrow Aspiration

The patient is lying flat and turned onto the hip. The bottom leg is extended straight, and the upper leg is bent at the knee. The patient is pre-pared and draped as usual for this procedure. The juncture of the sacroiliac region is palpated, and the finger is moved up away from the space over the broad crest of the posterior iliac wing. Following injection of local anesthesia, the biopsy needle is placed through the skin, over the iliac crest, and rotated 1 cm into the marrow space. The stylet is removed, syringe is attached, and bone marrow aspirate is taken.

Case No. 2. A 42-year-old female patient presented with partially edentulous maxilla. There was insufficient bone height of the right posterior maxilla for placement of implants. In addition, there was severe atrophy of the anterior maxilla. The treatment plan was sinus-lift subantral bone graft augmentation of the posterior maxilla. Bone graft of the anterior maxilla was to add sufficient bone for implants to support fixed crown and bridge restorations.

The atrophic recipient site was prepared and decorticated. There were two corticocancellous allograft bone blocks contoured to fit the anterior recipient site. Care was taken not to over contour the bone blocks at the expense of the cancellous bone. Transosseous lag screws stabilized the bone blocks for contouring and decortication, with small fissure burs, of its cortical surface. The bone blocks were removed and placed in an occluded syringe. The bone marrow aspirate was placed in the syringe covering the bone block. The plunger was placed, the syringe inverted to expel air, and the needle port again occluded. Pulling back on the plunger created a vacuum and saturated the bone blocks with marrow aspirate. Particulate resorbable matrix saturated with the marrow aspirate was mortised over the bone blocks. The mucoperiosteal flap was closed and sutured without tension on the incision.

### Sternum Bone Marrow Aspiration

In this awake patient, the area of the sternum below the suprasternal notch was shaved and prepared as described above. Subcutaneous local anesthesia was provided. Approximately one inch below the notch, the needle found the cortical plate of the sternum, and anesthesia was deposited under the periosteum. The length of needle passing from skin to making contact with bone is noted and recorded. The needle guard of the syringe was locked to expose the length of needle as noted when giving local anesthesia. This procedure was important so that the aspiration needle passes through only the outer cortex into the marrow space and not through the inner cortex into the aorta. The syringe was connected to the needle after the stylet was removed and bone marrow drawn into the syringe. The needle was twisted as it was removed from the bone, leaving a small puncture that was covered with a Band-Aid (Johnson & Johnson, Somerville, N.J.).

Case No. 3. A 52-year-old male presented with a partially edentulous mandible and failing right canine, fast bicuspid, and molar teeth. The indicated teeth were extracted, and the sockets debrided, irrigated and decorticated of the dental lamina. The porous resorbable matrix was saturated with bone marrow aspirate and placed in the sockets. The mucoperiosteal flap was repositioned and sutured without tension.

### Matrix

The ideal scaffold should mimic the extra-cellular matrix of autogenous bone. Preferably it is non-toxic, biocompatible, biodegradable at a rate that is compatible with bone remodeling without lowering the pH of surrounding tissues, has a porous structure of a geometry that permits cell ingrowth, and can be easily integrated into new bone. Further, the scaffold should have a micoporosity that supports and promotes angiogenesis and capillary in-growth.

A variety of tissue-engineered bone substitute materials are commercially available. Preferably, an osteoconductive scaffold is provided to serve as a delivery vehicle for the aspirated cells. This scaffold contains the cells within the graft. Because fast resorbing matrices may not remain at the recipient site for a long enough period to permit the full process of osteogenesis to unfold, a slowly resorbable, biodegradable, and biocompatible scaffold material may be provided. Porous, resorbable hydroxylapatite matrix, is an excellent material and produces satisfactory results. Beta-tricalcium phosphate is another material of choice for these procedures.

Preferably, the scaffold should possess both micro-porosity and macro-porosity with open-pore architecture and inter-connecting geometry to allow a large surface-to-area volume ratio. This will facilitate cell ingrowth and vascularization of the graft from the surrounding tissues. As is the case for all bone grafts, the graft material must be stabilized at the recipient site with at least one of screws, guided bone regenerative membranes, a titanium mesh and/or other suitable materials.

Cell transfer strategies of bone marrow aspirate stem cells to the scaffold delivers living cells to allografts, alloplasts, and xenografts or combinations thereof. Suitable allograft material includes demineralized bone an undemineralized bone. Suitable xenograft material includes bovine resorbable hydroxyapatie. Suitable alloplast material includes β-tricalcium phosphate, calcium sulfate and β-tricalcium sulfate, as well as combinations thereof. After the transplanted cells from the bone marrow differentiate, they provide the osteoblasts and osteoclasts that are needed for bone regeneration. When properly handled and administered, the transplanted cells lay down an initial unmineralized bone matrix, an osteoid, and initiate the mineralization process of laying the mineral component of bone, hydroxylapatite. When suspended into a suitable graft matrix the transplanted cells lay down the initial unmineralized bone, the osteoid, and initiate the mineralization process of proliferating the mineral component of bone, hydroxylapatite.

### Kit

In yet another embodiment of the invention, a kit for performing the presently contemplated inventive method is provided. Such a kit would include the following contents in a sterile-packaged tray.

| | | |
|---|---|---|
| 3 | Povidone Iodine Swab Sticks 1% | |
| | available iodine | |
| 2 | Absorbent Towels | |
| 1 | Fenestrated Drape | |
| 1 | “J” style bone marrow biopsy needle | |
| | 15 g x 10 cm | |
| 1 | 20 gauge x 1½″ | To draw the local anesthetic |
| 1 | 25 gauge x ⅝″ | Subcutaneous injection |
| 1 | 21 gauge x 1½″ | To the iliac crest |
| 1 | 5 ml syringe | For local anesthesia |
| 1 | Vial 5 cc lidoocaine 1% | For injection |
| 1 | 20 ml syringe | To collect bone marrow |
| 1 | Scapel blade with handle | Make stab incision through |
| | | skin |
| 3 | Gauze pads | |
| 1 | Bandage | |
| 1 | Specimen Cup | Place aspirate and graft |
| | | matrix |

### EXAMPLES

Certain embodiments of the present invention may be further understood by reference to the following specific examples. These examples and the terminology used herein is for the purpose of describing particular embodiments only and is not intended to be limiting.

Seven graft sites in five patients were evaluated. The xenograft scaffold was either PepCxen Putty (DENTSPLY Fria-dent CeraMed, Lakewood, Colo.) or C-Graft resorbable algae material (Clinician's Preference, Golden, Colo.). The alloplast scaffold was β-tricalcium phosphate (either Curasan AG, Kleinostheim, Germany or Vitoss; Malvern, Pa.). Three of the patients were grafted using sinus-lift subantral augmemtation. The sinus-lift surgery protocol was as follows.

Each patient was prepared and draped, then 2% Xylocalne with 1:200,000 epinephrine was locally injected. After adequate local anesthesia was achieved, a crestal incision and vertical relief incision were made. A mucoperiosteal flap was reflected along the crestal alveolar bone, superiorly to the height of the vestibule. The flap exposed the region of the canine fossa, malar buttress, and region of the pterygomaxillary fissure and the tuberosity. The procedure continued with the development of a quadrilateral osteotomy. After lifting the sinus membrane, the graft material (either peptide-enhanced anorganic bovine particles, resorbable algae-derived material, or β-tricalcium phosphate) was mixed with bone-marrow and inserted. The flap was then repositioned and sutured.

The fourth patient received an onlay particulate graft. For this procedure, a mucoperiosteal flap was relected exposing the labial cortical bone. Decortication of the labial plate was accomplished. A titanium mesh was secured with screws on the palate. A guided bone regenerative membrane was placed under the mesh, and under the membrane, a loose compaction of bone-marrow aspirate mixed with PepGen P-15 (DENTSPLY Friadent CeraMed) anorganic bovine putty was deposited. The titanium mesh was then secured with screws on the labial, and the periosteum was relieved to obtain primary closure without tension at the incision. The site was sutured with 3-0 Vicryl material (Ethicon, Inc., Johnson & Johnson, Somerville, M.

The fifth patient's left maxillary ridge in the canine-bicuspid area was grafted using a tunneling technique. A vertical mucoperiosteal incision was made, and a subperiosteal tunnel was developed. A composite graft consisting of bone-marrow aspirate mixed with loosely compacted C-Graft resorbable material was injected through the tunnel.

In each case, the bone marrow was extracted from the patient using the technique described above. This technique can be performed as an outpatient procedure with the patient under oral sedation and local anesthesia, intravenous sedation, or general anesthesia. The bone marrow can be extracted from the sternum, posterior ilium, or anterior iliac crest. A bone-marrow aspiration needle is held with the index finger near the tip to control the depth of penetration. The needle is then advanced with steady pressure and a twisting motion through the cortical bone to approximately 1 cm inside the marrow cavity. (Decreased resistance indicates penetration into the marrow cavity.) The obturator/stylet is removed, and a 10-syringe is attached to the needle. Following aspiration of 2-4 ml of bone marrow, the needle is removed.

All 5 patients were allowed to heal for 4-7 months. After obtaining informed consent, specimens of the grafted areas were taken with trephine drills. These were then submitted to an oral pathologist for standard histologic and histomorphogenic evaluation.

The analysis measured the percentage of graft material that had been converted into bone, percentage that was unresorbed, and remaining interstitial tissue. In addition, all bone identified in the biopsies was assessed to ascertain the percentage of bone that appeared to be vital.

### Results

### Case 1

After sinus-lift surgery to create the graft-recipient site, bone-marrow aspirate was mixed with C-Graft resorbable material and deposited with loose compaction to reconstitute the buccal wall of the maxilla. After 4 months, a biopsy was taken with a trephine drill from the crestal. bone to the superior aspect of the graft.

The percentage of the biopsy that was found to be bone was 31%. One hundred percent of that bone was vital. The percentage of unresorbed graft material within the core was 26%. Interstitial material constituted the remaining 43%. Within the cancellous bone pattern, all the graft particles were well incorporated.

New bone formation bridged the graft particles. Remodeling of mature bone was evident. New bone formation with C-Graft and the pattern of the coral were evident.

### Case 2

Bilateral sinus-lift surgery was initiated, and the right sinus was loosely filled with pure phase β-tricalcium phosphate mixed with bone-marrow aspirate to reconstitute the lateral wall, The left sinus recipient site was grafted with PepGen P-15 anorganic bovine particulate. After 4 months, biopsies were taken with a trephine drill from the crestal bone to the superior aspect of the grafts.

The percentage of the right-sinus (β-tricalcium phosphate) core biopsy that was found to be bone was 40%. One hundred percent of that bone was vital. The percentage of non-bone within the core was 3%. The remaining 57% of the sample consisted of stitial material. All the graft particles were well incorporated within the cancellous bone pattern. Good bone formation and distribution in the core biopsy was observed. The bone appeared to be very dense, with a good cancellous pattern incorporating well-formed connective tissue, and graft material particles. Tricalcium phosphate particles were embedded in the newly formed bone with evidence of osteoid and osteoblasts.

The percentage of the left-sinus (PepGen P-15) core biopsy that was found to be bone was 14%, with 100% of that bone found to be vital. The percentage of non-bone within the core was 36%. The tight compaction of the graft material that was evident suggests an explanation for the decreased bone formation. Tight compaction of the scaffold leads to a decrease in space between the particles and a decrease in bone formation between the particles. New bone formation with osteoid and osteoblasts and PepGen P-15 interaction was observed with the bone. The PepGen P-15 particles were both embedded in and attached to the bone.

### Case 3

Bilateral sinus-lift surgery was initiated, and both sinuses were grafted with β-tricalcium phosphate (Vitoss) mixed with bone-marrow aspirate. After four and one half months, biopsies were taken with a trephine from the crestal bone to the superior aspect of both grafts. The percentage of the right-sinus biopsy that was found to be bone was 23%, with 89% of that bone found to be vital. The percentage of non-bone within the core was 13%. The percentage of the left-sinus biopsy found to be bone was 16%, with 86% of the bone found to be vital.

New bone formation and resorbing graft matrix within the right biopsy was observed. Viable cells within the lacunae of new bone formation were observed in the biopsy core.

### Case 4

This core was taken from the anterior maxilla particulate onlay graft with PepGen P-15 after four months of healing. The percentage of the biopsy that was found to be bone was 32%, with 100% of that found to be vital. The percentage of non-bone identified within the core was 15%. The remaining 53% consisted of interstitial material.

The core showed a sound cancellous bone pattern with new bone formation independent of the particles of PepGen P-15. A few particles were incorporated into the new bone formation. Close inspection showed new bone formation attached to the PepGen P-15 particles.

### Case 5

After 7 months of healing, a biopsy core was taken and found to consist of 45% newly formed bone. All of that bone was vital, and there was no residual graft material present. The cancellous bone pattern was composed of very thick trabeculae. New bone formation with C-Graft and the pattern of the scaffold was visible upon microscopic inspection.

### Results Summary

Bone-graft healing involves the recruitment and proliferation of cells capable of restoring the graft site to its original form and function. A potential cause of variation in the results obtained when using stem cells may be the age of the patient. As patients age, their red marrow (rich in stem cells) decreases and is replaced by yellow marrow (poor in stem cells). Techniques for concentrating the stem cells for older patients are now being evaluated.

In four of the seven cases included in the current study, the percentage of mineralized bone identified (between 31% and 45%) compares favorably with the mean percentage of mineralized bone found by Szabo et al. (38.34%) (Szabo G, Huys L, Coulthard P. A prospective multicenter randomized clinical trial of autogenous bone versus β-tricalcium phosphate graft alone for bilateral sinus elevation: Histologic and histomorphometric evaluation. Int J. Oral Maxilofac Implants. 2005; 20:371-381) in sites grafted with autogenous bone. As noted above, the use of stem cells aspirated from bone marrow eliminates the need for the creation of a second surgical site for harvesting autogenous bone.

This study demonstrates that the combination of stem cells (derived from bone marrow) with a scaffold material within a bones grafting site can produce a significant quantity of viable bone. The therapeutic potential of bone regeneration by such cell-based strategies is highly promising. Stem cells derived from bone marrow are remarkably plastic and pluripotent, and they can roam throughout the body, taking up residence wherever they are needed.

### CONCLUSION

The addition of bone marrow aspirate to an osteoconductive matrix can contribute substantially to the efficacy of bone augmentation in the maxilla and mandible. The bone marrow fosters osteogenesis by providing stems cells that can differentiate into bone-producing osteoblasts. Growth factors contained in the marrow further enhance the osteoinductive process. With the current knowledge about the multipotency of adult bone marrow and availability of advanced, bioengineered bone graft substitutes, the invention provides a new standard for bone grafting.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A bone grafting method comprising the steps of:
harvesting a bone marrow aspirate from a patient, said bone marrow aspirate containing at least one type of cells selected from the group consisting of osteoblasts, adult stem cells and primitive mesenchymal cells;
combining said cells from the bone marrow aspirate with a suitable graft material; and
securing the graft material at a site in a patient to receive a graft.

2. The method of claim 1, wherein said graft material is selected from the group consisting of an allograft, xenograft or alloplast matrix material.

3. The method of claim 1, wherein said bone marrow aspirate further contains cytokines and growth factors.

4. The method of claim 3, where said cytokines and growth factors are provided from plasma from the bone marrow aspirate.

5. The method of claim 1, where said step of harvesting the bone marrow aspirate includes the step of centrifuging the bone marrow aspirate to separate the aspirate into multiple layers.

6. The method of claim 5, further comprising the step of providing the plasma from the bone marrow aspirate with the cells from the bone marrow aspirate and the suitable graft material.

7. The method of claim 1, further comprising the step of filtering the bone marrow aspirate to separate the at least one type of cells selected from the group consisting of osteoblasts, adult stem cells and primitive mesenchymal cells from the bone marrow aspirate.

8. The method of claim 7, wherein said filtering also separates the plasma from the bone marrow aspirate.

9. A bone graft produced in accordance with the method of claim 1.

10. A kit for bone grafting comprising at least one of each of the following, provided in a suitable sterile container:
povidone iodine swab sticks 1% available iodine
absorbent towels
fenestrated drape
“J” style bone marrow biopsy needle 15 g×10 cm
20 gauge×1½″ needle
25 gauge×⅝″ needle
21 gauge×1½″ needle
5 ml syringe
vial 5 cc lidoocaine 1%
20 ml syringe
scapel blade with handle make stab incision through skin
gauze pads
bandage and
specimen cup.
